(19) United States
(12) Patent Application Publication
(10) Pub. No.: US 2026/0253169 A1
(43) Pub. Date: Aug. 27, 2026

(54) ENHANCED ZOOM FUNCTIONALITY FOR EXTENDED REALITY (XR) SYSTEMS USING PASS-THROUGH TECHNOLOGY

(71) Applicant: Covidien LP, Mansfield, MA (US)

(72) Inventor: Lavie P. Golenberg, Singapore (SG)

(73) Assignee: Covidien LP, Mansfield, MA (US)

(21) Appl. No.: 19/455,098

(22) Filed: Jan. 21, 2026

Related U.S. Application Data
(60) Provisional application No. 63/761,625, filed on Feb. 21, 2025.

(51) Int. Cl. G06T 3/40; G02B 27/00; G02B 27/01; G06F 3/04845

(52) U.S. Cl. CPC G06T 3/40; G02B 27/0093; G02B 27/017; G06F 3/04845; G02B 2027/0138; G06F 2203/04806

(57) ABSTRACT

An extended reality head-mounted display system is provided. The extended reality head-mounted display system includes: a head-mounted display having a lens and a display screen located on an inner surface of the lens for projecting a real-world video feed; integrated zoom-in functionality; and a processor coupled to memory. The processor is configured to execute instructions that cause a portion of the display screen to project a zoomed-in image of the real-world video feed to a wearer of the head-mounted display. An extended reality head-mounted display system including multiple head-mounted displays is also provided, as is a method for zooming-in on a portion of the real-world video feed using the extended reality head-mounted display system.

### CROSS-REFERENCE TO RELATED APPLICATIONS

This application claims the benefit of U.S. Provisional Application No. 63/761,625 filed Feb. 21, 2025, entitled “Enhanced Functionality for Extended Reality (XR) Systems Using Pass-Through Technology,” which is incorporated herein by reference in its entirety.

### BACKGROUND

Extended Reality (XR) systems, which can include virtual reality (VR), augmented reality (AR), and mixed reality (MR) applications, can be used, for example, in the medical field for training or simulation, including in a remote context, enhanced visualization during surgical procedures, tracking of surgical instruments or medical devices, assessment of patient vitals, and many other aspects of patient care. Such XR systems can include a head-mounted display (HMD), such as a headset or glasses, a controller, sensors, a camera, an external display, a controller, an audio system, a haptic feedback system, various inputs and outputs, and other components that facilitate an immersive experience for the user.

In extended reality (XR) systems, pass-through technology refers to the ability to see the real-world environment while wearing a head-mounted display (HMD). Pass-through technology can facilitate the creation of mixed reality (MR) experiences, where generated images can be superimposed on, be displayed relative to, or interact with real objects in the real world physical environment in a seamless way.

For example, pass-through technology can use cameras or sensors on the HMD (e.g., on the outside of the HMD) to capture video footage of the physical environment and display it on a lens or an internal screen of the HMD, allowing a user to still have a sense of his or her surroundings. Pass-through technology is often used in XR systems to provide a user with a “window” into the real world to safely navigate his or her environment while still being immersed in virtual content.

Despite advances in the camera and optical systems used with pass-through technology in XR systems, resulting in improved camera resolution (e.g., 4K and 8K resolution), the ability for a user to “zoom in” to his or her surroundings with high resolution, similar to using binoculars in the physical environment, has not been possible with existing systems.

As such, a need exists for an improved XR system that allows for enhanced zoom functionality in a seamless way while utilizing pass-through technology.

### SUMMARY

In accordance with one embodiment of the present disclosure, an extended reality (XR) head-mounted display system is provided. The extended reality head-mounted display system also includes a head-mounted display may include a lens and a display screen located on an inner surface of the lens for projecting a real-world video feed; integrated zoom-in functionality; and a processor coupled to memory, where the processor is configured to execute instructions that cause a portion of the display screen to project a zoomed-in image of the real-world video feed to a wearer of the head-mounted display.

The extended reality head-mounted display system may include an input configured to allow the wearer of the head-mounted display to activate the integrated zoom-in functionality. The one or more inputs may include a microphone, a physical actuator, or a combination thereof. The head-mounted display further may include an eye-tracking sensor. The camera can provide the real-world video feed to the display screen in real time. The camera can also be a part of the head-mounted display. Alternatively, the camera can be an external camera that is separate from the head-mounted display. A periphery of the display screen can be configured to display the real-world video feed when the integrated zoom-in functionality is activated. The real-world video feed can, in some aspects, include a surgical procedure. The zoomed-in image can capture a surgical procedure being performed on a patient, one or more vital signs of a patient, a surgical environment, or a combination thereof.

In another aspect, an extended reality head-mounted display system is provided. The extended reality head-mounted display system includes a first head-mounted display that includes a first lens and a first display screen located on an inner surface of the first lens for projecting a first real-world video feed. The system also includes a first integrated zoom-in functionality associated with the first head-mounted display. The system also includes a first processor coupled to a first memory, where the first processor is configured to execute instructions that cause a portion of the first display screen to project a first zoomed-in image of the first real-world video feed to a first wearer of the first head-mounted display. The system also includes a second head-mounted display may include a second lens and a second display screen located on a second inner surface of the second lens for projecting the first real-world video feed. The system also includes a second integrated zoom-in functionality associated with the second head-mounted display. The system also includes a second processor coupled to a second memory, where the second processor is configured to execute instructions that cause a portion of the second display screen to project a second zoomed-in image of the first real-world video feed to a second wearer of the second head-mounted display.

In one aspect, the first zoomed-in image of the first real-world video feed on the first display screen may not be visible to the second wearer of the second head-mounted display, and the second zoomed-in image of the first real-world video feed on the second display screen may not be visible to the first wearer of the first head-mounted display. The extended reality head-mounted display system may include one or more inputs configured to independently allow the first wearer of the first head-mounted display to activate the first integrated zoom-in functionality on the first head-mounted display and the second wearer of the second head-mounted display to activate the second integrated zoom-in functionality on the second head-mounted display.

In another aspect, the first zoomed-in image of the first real-world video feed may be visible to the second wearer so that the second zoomed-in image is a duplicate of the first zoomed-in image of the first real-world video feed.

The first head-mounted display, the second head-mounted display, or both further may include an eye tracking sensor. The first camera can project the first real-world video feed to the first display screen, the second display screen, or both in real time. The extended reality head-mounted display system may include a second camera that is part of the second head-mounted display. In addition, a periphery of the first display screen can be configured to display the first real-world video feed when the first integrated zoom-in functionality is activated, and a periphery of the second display screen can be configured to display the first real-world video feed when the second integrated zoom-in functionality is activated.

The present disclosure also contemplates a method for zooming-in on a particular portion of a video feed. For example, the method includes receiving, via a first head-mounted display including a first processor, a real-world video feed; projecting, via the first head-mounted display, the real-world video feed onto a first display screen located on a first inner surface of a first lens of the first head-mounted display; and receiving, via a first wearer of the first head-mounted display, a first input that causes the first processor to execute instructions to zoom-in on a portion of the real-world video feed being projected on the first display screen.

The method also includes receiving, via a second head-mounted display including a second processor, the real-world video feed; projecting, via the second head-mounted display, the real-world video feed onto a second display screen located on a second inner surface of a second lens of the second head-mounted display; and receiving, via a second wearer of the second head-mounted display, a second input that causes the second processor to execute instructions to zoom-in on a portion of the real-world video feed being projected on the second display screen.

Other features and aspects of the present disclosure are set forth in greater detail below.

### BRIEF DESCRIPTION OF THE DRAWINGS

A full and enabling disclosure of the present disclosure, including the best mode thereof to one skilled in the art, is set forth more particularly in the remainder of the specification, including reference to the accompanying figure, in which:

FIG. 1 is a block diagram of an extended reality (XR) head-mounted display system for use in a real-world environment, such as a surgical environment, as contemplated by the present disclosure;

FIG. 2 is a perspective view of an XR head-mounted display contemplated by the present disclosure;

FIG. 3 is a schematic view of an XR head-mounted display system illustrating the pass-through technology utilized in an XR head-mounted display contemplated by the present disclosure;

FIG. 4 illustrates a portion of a real-world environment, such as a surgical environment, in which the system of the present disclosure can be utilized, where real-world video of the surgical environment is displayed via a screen on a lens of a head-mounted display contemplated by the present disclosure;

FIG. 5 illustrates a display screen on an inner surface of two lenses of a head-mounted display, where a portion of one lens includes a zoomed-in image of the surgical environment that is displayed as a result of the integrated zoom-in functionality built into the display screen;

FIG. 6 is a block diagram of an XR system for use in multiple real-world environments, such as a surgical environment and a remote location outside of the surgical environment, as contemplated by the present disclosure;

FIG. 7 illustrates a display screen on an inner surface of two lenses of a first head-mounted display, where both lenses show a live, real-world video without zoom-in from the perspective of the wearer of the first head-mounted display or a wearer of a second head-mounted display;

FIG. 8 illustrates a display screen on an inner surface of two lenses of a first head-mounted display, where both lenses show a live, real-world video in real-time without zooming-in from the perspective of the wearer of the first head-mounted display or a wearer of a second head-mounted display, where a portion of the display screen with zoom-in functionality on each lens is shown in dashed lines, and a periphery outside the portion of the display screen with zoom-in functionality shows the live, real-world video in real time without zooming-in;

FIG. 9. illustrates a display screen on an inner surface of two lenses of a first head-mounted display, where both lenses show a live, real-world video in real-time without zooming-in from the perspective of the wearer of the first head-mounted display or a wearer of a second head-mounted display about a periphery of the display screen, while the portion of the display screen with zoom-in functionality on each lens is shown in dashed lines and includes a zoomed-in image; and

FIG. 10 illustrates a block diagram of a computer system according to one or more aspects of the present disclosure.

Repeat use of reference characters in the present specification and drawing is intended to represent the same or analogous features or elements of the present disclosure.

### DETAILED DESCRIPTION

Embodiments of the present disclosed extended reality (XR) head-mounted display system are described in detail with reference to the drawings, in which like reference numerals designate identical or corresponding elements in each of the several views.

As will be described in detail below, the present disclosure is directed to an extended reality system including one or more head-mounted displays that can have independent zoom-in functionality to allow different wearers observing the same real-world video feed to focus in on unique, independent portions of the real-world video feed. However, it is also to be understood that in other embodiments, the zoom-in functionality on multiple screens can be identical duplicates of each other, which can be desirable for training, communication, and validation purposes.

The system can include one or more head-mounted displays. At least one of the head-mounted displays includes a camera for recording and transmitting real-world video to a display screen on the one or more head-mounted displays. The real-world video feed can be recorded from a first wearer's perspective in real-time. The video feed can be displayed on one or more screens embedded in or otherwise integrated into one or more lenses of the wearer's head-mounted display. The video feed can also be displayed on one or more display screens embedded in or otherwise integrated into one or more lenses of a head-mounted display of a second wearer. The second wearer can be located remotely from the first wearer. The head-mounted display of the first wearer can be configured to receive an input (e.g., voice or physical activation, touch activation, etc.) from the first wearer, and the input can provide instructions to the first head-mounted display to zoom-in on a portion of the real-time video feed being displayed on the one or more display screens on the first head-mounted display. Further, the head-mounted display of the second wearer is configured to receive an input (e.g., voice or physical activation, touch activation, etc.) from the second wearer, and the input can provide instructions to the second head-mounted display to zoom-in on a portion of the real-time video being displayed on the one or more display screens on the second head-mounted display.

It should be understood that the zoom-in functionality of the first head-mounted display can be completely independent of the zoom-in functionality of the second head-mounted display such that the first wearer's video feed is not altered or zoomed-in when the second wearer is zooming in on the first wearer's video feed on his or her own, independent display screen on the second head-mounted display. This feature allows the various wearer's to control which portion of the real-world video feed they would like to see in more detail. Further, the zoom-in functionality can be displayed on only a portion of the one or more display screens on each of the head-mounted displays such that a portion of the one or more display screens continues to show the real-world video feed in real time without any zoom-in functionality applied thereto.

Moreover, it should also be understood that in some embodiments, the zoom-in functionality on the second head-mounted display can be dependent on or linked to the zoom-in functionality of the first head-mounted display such that the first wearer's video feed is zoomed-in on the second head-mounted display identically to how the first wearer's video feed is being zoomed-in on the first head-mounted display. This feature can be useful in training sessions, such as when a surgeon wants to communicate with an observer to explain a procedure, anatomical structure, etc. where it would be beneficial for the surgeon and observer to have the same view.

The system can be used in surgical environments, remote training sessions, and in outdoor/indoor environments to allow the wearer of each individual head-mounted display to control the portion of a real-time video that the wearer would like to magnify for the individual wearer, regardless of the source of the real-time video, which could be the camera on the wearer's head-mounted display or a camera on a different wearer's head-mounted display. For example, a remote person observing a medical procedure could zoom-in on a particular anatomical structure of interest from a real-time video taken from a surgeon's perspective, or a surgeon performing a medical procedure could zoom-in on an optical tower or surgeon console in the operating room to more easily observe a patient's vital signs or other information.

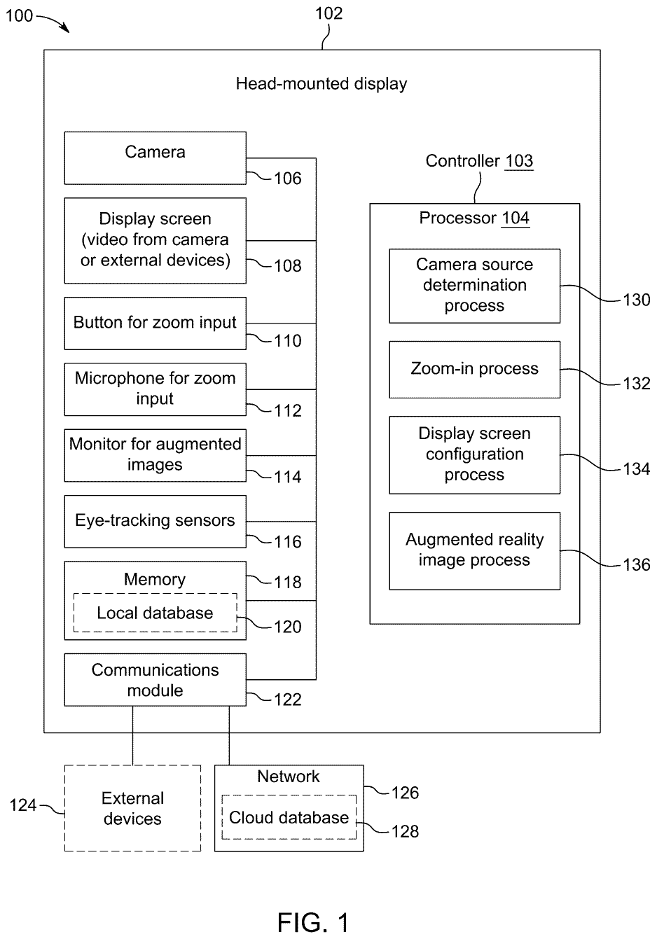

Turning to FIG. 1, the XR head-mounted display system 100 can include a head-mounted display 102 that can be placed on a wearer's head. The XR head-mounted display system 100 can also include a controller 103 and a processor 104 for executing various instructions based inputs, data, and other information provided by the different components of the system 100, as will be discussed in more detail below. Briefly, the XR head-mounted display 102 includes memory 118 that can store instructions that are executed by the processor 104.

In addition, the XR head-mounted display system 100 can include a camera 106. The camera 106 can have a field-of-view (FOV) that overlaps with the natural field-of-view of the wearer's eyes when the head-mounted display 102 is worn. The camera 106 records a real-world view of the wearer's surroundings in real time. Data (e.g., video feed and/or still images) from the camera 106 can be sent to a display screen 108 on a lens of the head-mounted display 102. The display screen 108 can also receive real world or augmented images from one or more additional cameras that are distinct from the camera 108 that is integrated into the head-mounted display 102. For instance, the display screen 108 may present augmented reality data (e.g., images, graphics, text, icons, etc.) on a portion of a lens (or lenses) of the head-mounted display 102 so that a wearer may view the augmented reality data as the wearer looks through a lens of the head-mounted display 102. In this way, the augmented reality data can overlap with the wearer's view of the real-world environment from the video feed associated with camera 106 and/or other external cameras. Further, one or more additional external cameras (not shown) may be placed in one or more locations in the real-world environment including, but not limited to, above the operating table, on surgical tools, on various portions of a robotic surgical system, etc. when the real-world environment is a surgical environment. In this way, the head-mounted display 102 can display real-world video from multiple sources as well augmented images to a clinician or others, who may be in the room or at a remote location, during a surgical procedure.

Referring still to FIG. 1, the images captured by the camera 106 and/or external cameras can be provided to the controller 102 to provide a comprehensive, real-world view of a surgical environment which is provided to the head-mounted display 102. The comprehensive overview may provide a broad range of insights to assist the one or more clinician in carrying out an efficient and safe surgical procedure or to help train other individuals who are wearing their own head-mounted displays. The controller 102 can include a transceiver configured to receive video, still frame images, or data. In some embodiments, the transceiver can include an antenna to receive the video, still frame images, or data via a wireless communication protocol. The video, still frame images, or data can be provided to the processor 104. The processor 104 can include an image processing filter that processes the received video, still frame images, or data to generate a zoomed-in image or video feed and/or an augmented image or video.

Referring again to FIG. 1, the XR head-mounted display system 100 can include a physical actuator 110, such as a button, knob, dial, touch-screen, switch, and the like that can be activated by the wearer, such as by turning, pushing, touching, and the like, to indicate to the controller 103 that the wearer desires to zoom-in on a portion of the real-world video feed that is being displayed on the display screen 108. In response, instructions can be executed by the processor to zoom-in on the video feed being displayed.

As shown in FIG. 1, the XR head-mounted display 102 can include a microphone 112 configured to receive voice commands from a wearer of the head-mounted display 102. In particular, in response to the voice commands sensed by the microphone and received by the controller, the processor can execute instructions to zoom-in on the video feed being displayed. For example, voice recognition software and natural language processing can be used to facilitate the ability of the head-mounted display 102 to process and interpret the voice commands captured by the microphone 112 and to interpret complex voice commands based on the context of the surrounding environment.

The XR head-mounted display system 100 can also include a monitor 114 that is also present on the lens but separate from the display screen 108 to display the aforementioned augmented reality data (e.g., images, graphics, text, icons, etc.) to the wearer. Additionally or alternatively, it should be understood that the augmented images can also be overlaid or superimposed on the real-world video feed and/or the zoomed-in portion of the real-world video feed from the camera 106. It should also be understood that the augmented images can be overlaid or superimposed on real-world video feed and/or the zoomed-in portion of the real-world video feed from an external camera that is not part of the XR-head-mounted display system 100 depending on which video feed or images are being viewed by the wearer at the time.

Referring still to FIG. 1, the head-mounted display 102 of the system 100 can further include an eye-tracking sensor 116 configured to sense attributes (e.g., pupil position) of an eye (or eyes) of a wearer of the head-mounted display 102 and track eye movement of the wearer. The eye-tracking sensor 116 can include a right-eye camera and a left-eye camera. The right-eye camera and the left-eye camera can be located in a lens portion of the head-mounted display 102 so that a right field-of-view of the right-eye camera includes the right eye of the wearer and a left field-of-view of the left-eye camera includes the left eye of the wearer of the head-mounted display 102.

The attributes may be processed to determine a direction or point at which the wearer of the head-mounted display 102 is looking (i.e., a gaze of the wearer). This allows for an interaction between the user and the environment to be understood and can aid in the zooming-in of the portion of an environment, object, portion of an image, etc. that is of interest to the wearer. The eye-tracking sensors 116 can emit light that is reflected off of the eye and detected by the camera 106 or any other optical sensor. The detected reflected light is analyzed by the controller 102 to extract eye rotation from changes in reflections. In some embodiments, the controller 102 can use corneal reflection and the center of the pupil as features to track over time. In other embodiments, reflections from the front of the cornea and the back of the lens can be used to track eye movement. In yet other embodiments, features from inside the eye, e.g., the retinal blood vessels, can be followed as the eye rotates. These methods of eye tracking are capable of tracking the gaze of the wearer so that the controller 102 may determine a location of interest for the wearer 154, such as an area that the wearer 154 would like to zoom-in on for closer inspection.

The system 100 can further include a communications module 122 to facilitate communication between the controller 103 and various external devices 124. The communications module 122 can include various circuits (i.e., modules) configured to communicate in a variety of wireless protocols. For example, the wireless module may include ultra-wideband (UWB) module, a Wi-Fi module, and/or a Bluetooth module. The communications module 122 can be configured to wirelessly couple the head-mounted display 102 to various external device(s) 124, such as, but not limited to additional head-mounted displays, tablets, mobile computing devices, laptops, desktop computers, surgeon consoles, towers, surgical instruments (e.g., endoscopes or catheters), and the like, which can each include one or more cameras of their own to record video in real time, and/or to a network 126 (i.e., cloud) in order to exchange data. For example, the external device(s) 124 can include a computer system such as computer system 300, discussed in more detail below, that, through a wireless communication link, can help process data from the head-mounted display 102. In another example, the network 126 can include a cloud database 128 that, through a wireless communication link, can help store and retrieve data with the head-mounted display 102. The communications module may also be able to determine a position of the head-mounted-display 102 relative to an external device 124. For example, an UWB module may be able to determine a relative range between two devices using a round trip time (RTT) of a signal in a communication between the two devices. Further, when the UWB module includes an array of receivers, a relative direction between the two devices may be determined based on a times of arrival of the signal at the receivers. Accordingly, data from the communications module 122 can be used to help determine the direction and/or position of a device or person in the global environment.

Further, the processor 104 can be configured by software to perform a plurality of processes required for interaction between the video feed received by the head-mounted display 102 and displayed to a wearer, as well as a plurality of processes required for interaction between the video feed received by the head-mounted display 102 and displayed to other users, such as users wearing their own, individual head-mounted displays 102, and/or a plurality of processes required for interaction between video feed from an external camera (e.g., a camera on an surgical instrument such as an endoscope or catheter) and the wearer of the head-mounted display 102. The plurality of processes can include a camera source determination process 130, a zoom-in process 132, a display screen configuration process 134, and/or an augmented reality image process 136. The plurality of processes may be embodied as programs stored in (and retrieved from) a memory 118 (e.g., from a local database 120). The disclosed approach can combine data and/or functions from these processes to provide an individualized wearer experience when viewing a video feed on a display screen 108 of a lens of the head-mounted display 102.

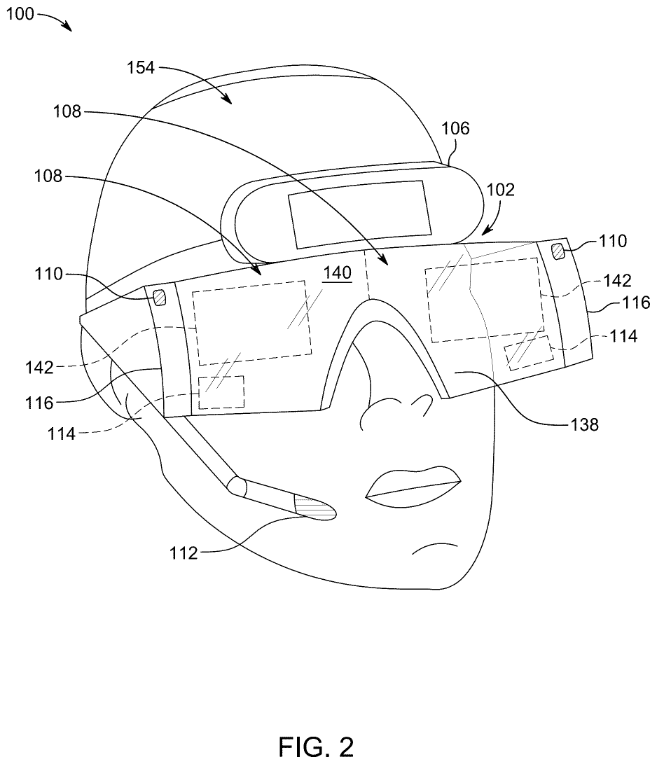

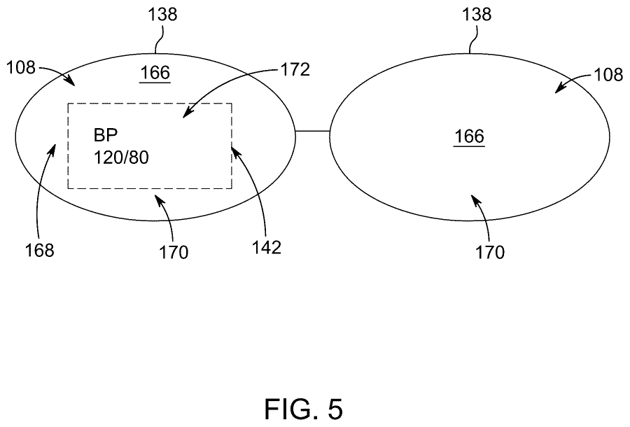

FIG. 2 is a perspective view of the XR head-mounted display system 100 that is described above in FIG. 1. As shown in FIG. 2, the XR head-mounted display system 100 includes a head-mounted display 102 that can be worn by a wearer 154. The head-mounted display 102 can be configured to be worn on a head and face of the wearer 154 via earpieces and a frame. The head-mounted display 102 can include a lens 138 having an outer surface 140 and an inner surface 166 (see FIGS. 5 and 7-9) that can be positioned in front a left eye of the user and a right eye of the user as a single lens or as two lenses. The portions of the head-mounted display 102 can be collectively referred to as the frame of the head-mounted display 102. The frame of the head-mounted display 102 can contain electronics to enable function. For example, the frame may include a battery, the processor 104, memory 118 (e.g., non-transitory computer readable medium), and electronics to support sensors (e.g., camera 106, eye-tracking sensor 116, etc.), and interface devices (e.g., display 108, physical actuator 110, microphone 112, monitor 114, network adapter, etc.). As shown, the camera 106 can be integrated with the head-mounted display 102 to provide a real-world view of video transmitted in real time from the user's point of view. Videos and/or images captured by the camera 106 can be interpreted by the controller 102 and can optionally be augmented before being displayed on a display screen 108 present on an inner surface of the lens 138. Further, a portion 142 of the display screen 108 of the head-mounted display 102 can include zoom-in functionality to allow the wearer 154 to focus on particular areas of interest in the real-world environment from the video feed obtained from the camera 106 or from video feed obtained from external devices 124.

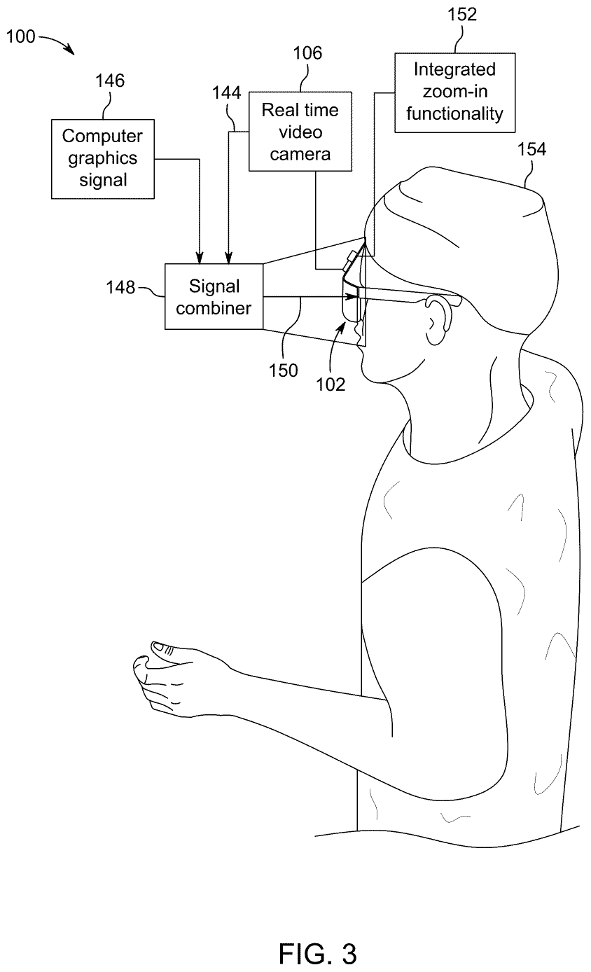

Turning now to FIG. 3, a schematic view of an XR head-mounted display system 100 illustrating the pass-through technology utilized in an XR head-mounted display 102 contemplated by the present disclosure. As shown, the head-mounted display 102 can be positioned in front of the eyes of the wearer 154. The head-mounted display 102 includes integrated zoom-in technology that is built into an inner surface of a lens of the head-mounted display 102 to allow a video feed from a display screen on the lens to be digitally zoomed-in upon receiving an input from the wearer 154. However, it should also be understood that the zoom-in technology can be effectuated via an optical zoom platform as well. In any event, the XR head-mounted display system 100 of the present disclosure facilitates zooming-in of a video feed that is projected on the display screen 108 (see FIGS. 2 and 7-9). In particular, a camera 106 that is part of the head-mounted display 102 can record real-world video in real time that is then passed through to the display screen via a video signal 144. It should also be understood, however, that the video feed can originate from an external camera in some embodiments. In additional embodiments, a computer graphics signal 146 can be combined with the video signal 144 via a signal combiner 148 so that a combined real-world video and computer graphics signal 150 can be passed through to the display screen 108 so that the wearer 154 can observe a video feed of a real-world environment around him or her with augmented reality images, graphics, text, icons, etc. overlaid or superimposed on the video feed.

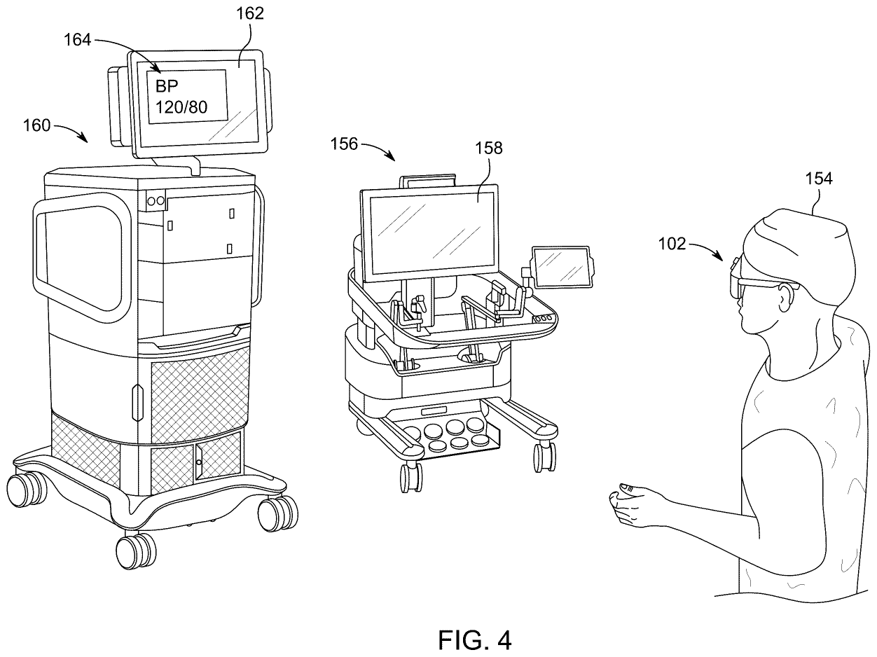

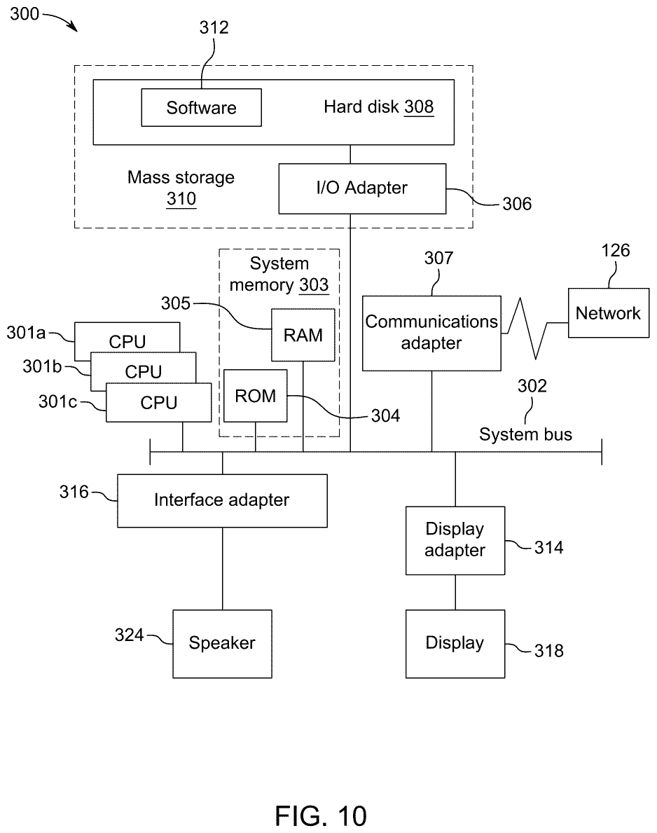

Next, FIG. 4 illustrates a portion of a real-world environment, such as a surgical environment, in which the XR head-mounted display system 100 of the present disclosure can be utilized, where real-world video of the surgical environment is displayed via a screen on a lens of a head-mounted display 102. As shown, the environment can include a surgeon console 156 having a display 158, a tower 160, which can include computer system 300 (see FIG. 10), and a tower display 162. Within the surgical environment, the wearer 154, which can be a surgeon or other medical professional, may need to see an area of interest 164 more clearly. In the particular environment shown in FIG. 4, the area of interest 164 an area on the tower display 162 that corresponds to a patient's vital signs. However, it is to be understood that the area of interest 164 could be any area of interest, such as, but not limited to, an area of interest in a field of view of a patient's anatomy during a surgical procedure, an image on a camera on an endoscope, an x-ray image, an ultrasound image, an MRI image, or any other imaging of a patient, patient records on a display screen, etc.

Referring to FIG. 5, once an area of interest 164 has been identified by the wearer 154 as an area where closer inspection is needed, the wearer 154 can initiate the head-mounted display 102 to begin a zoom-in process on the area of interest 164 via the physical actuator 110 by activating the physical actuator button and indicating a particular zoom factor (e.g., 1.5×, 2×, 5×, etc.) and/or the microphone 112 by, for example, speaking instructions such as, “Zoom-in at location L at 5× resolution.” Further, a natural language model can be used to interpret the instructions sensed by the microphone 112 so that the processor 104 can execute the zoom-in process 132 accurately. Alternatively or additionally, the eye tracking sensors 116 can be used to determine the area of interest 164 where zooming-in is desired. illustrates a display screen on an inner surface of two lenses of a head-mounted display, where a portion of one lens includes a zoomed-in image of the surgical environment that is displayed as a result of the integrated zoom-in functionality built into the display screen.

In any event, the zoomed-in image 172 of the area of interest 164 can then be projected onto the display screen 108 on inner surface 166 of the lend 138 of the head-mounted display 102. Further, a periphery 168 of each display screen 108 present on each lens 38 of the head-mounted display 102 can continue to show the real-world feed 170 in its original, un-zoomed format to act as a safety measure to ensure that the wearer 154 remains aware of his or her real-world surroundings. Further, although only one zoomed-in image 172 is shown in FIG. 5, it should be understood that both inner surfaces 166 of both lenses 138 of the head-mounted display 102 can include one or more portions 172 of the display screen 108 that have zoom-in functionality, and if a single lens 138 is utilized in the head-mounted display, the lens 138 can include one or more portions that have zoom-in functionality.

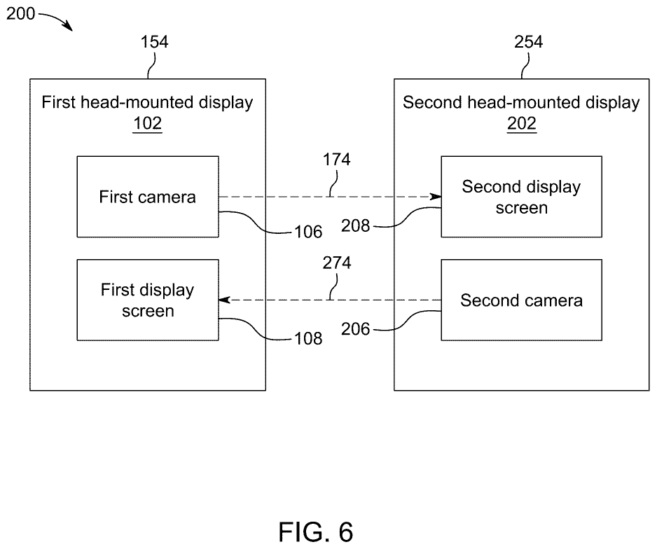

As referenced above and referring to FIG. 6, the present disclosure contemplates an XR head-mounted display system 200 that includes more than one head-mounted display worn by more than one user. Such an embodiment facilitates, for example, the ability to train personnel or proctor procedures in remote settings outside the surgical environment and in a different location entirely from a surgical suite. The XR head-mounted display system can include a first head-mounted display 102 worn by a first user 154 and a second head-mounted 202 worn by a second user 254. The first camera 106 associated with the first head-mounted display can record a video feed 174 of the real-world environment from the perspective of the first wearer 154 that can then be projected onto the display screen 208 of the second head-mounted display 202. Likewise, the second camera 206 associated with the second head-mounted display 202 can record a video feed 274 of the real-world environment from the perspective of the second wearer 254 that can then be projected onto the display screen 108 of the first head-mounted display 102. However, it should also be understood that it is not required that each head-mounted display 102 or 202 project video feeds from the other of the head-mounted displays 102 or 202, and, instead, each head-mounted display 102 or 202 may be used in some situations to only project the video feed 174 or 274 received only from that specific camera 106 or 206. In still other embodiments, the video feed projected onto each of the head-mounted displays 102 or 202 can come from another external source, such as another head-mounted display, a tablet, a mobile computing device, a laptop, a desktop computer, a surgeon console, a tower, a surgical instrument (e.g., endoscope or catheter), and the like. Further, regardless of where the video feed originates that is projected to each individual display screen, it should be understood that the zoom-in functionality of each display screen is completely individual to each head-mounted display such that a second wearer zooming in on a video feed recorded from the perspective of the first wearer does not alter the video feed seen by the first wearer (e.g., the video feed seen by the first wearer on the display screen of the first head-mounted display does not get zoomed-in when the second wearer initiates the zoom-in functionality on the display screen of the second head-mounted display).

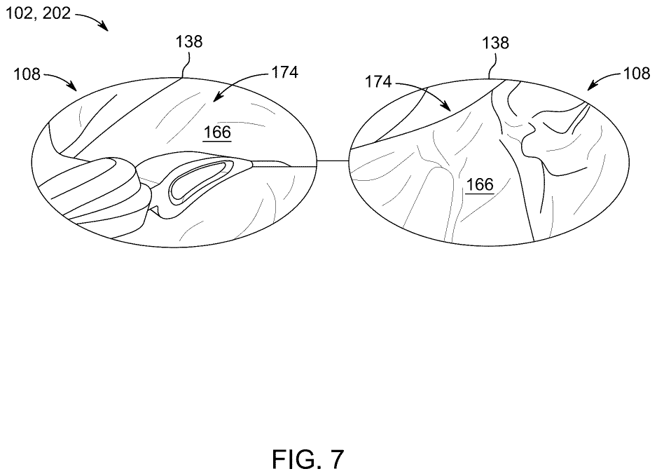

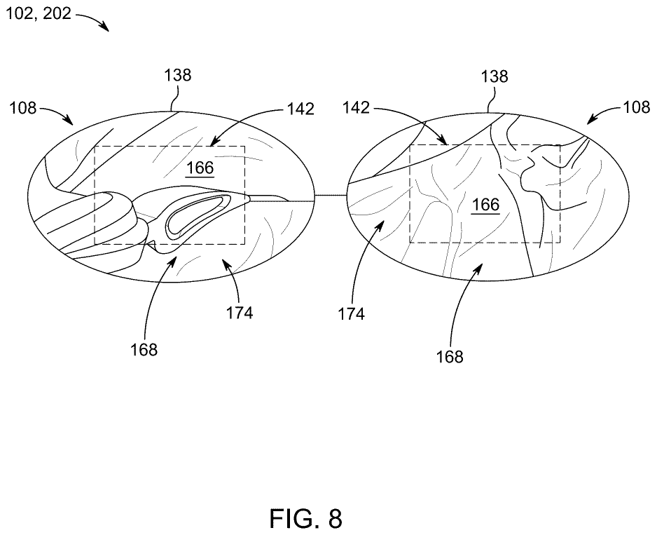

Turning now to FIGS. 7-9, a real-world, real time video feed 174 in real time of an anatomical surgery site is displayed on a head-mounted display 102 or 202 without any zoom-in functionality activated. The video feed 174 is shown on the inner surface 166 of the lens 138 of the head-mounted display 102 or 202. FIG. 7 illustrates a display screen on an inner surface of two lenses of a first head-mounted display, where both lenses show a live, real-world video in real time without zoom-in from the perspective of the wearer of the first head-mounted display or a wearer of a second head-mounted display. Next, FIG. 8 illustrates the portion 142 of the display screen 108 having zoom-in functionality, prior to the zoom-in functionality being activated. Further, a periphery 168 of the display screen 108 is shown as well, where the periphery 168 displays the real-world, real time video feed 174 in its original format to serve as a safety measure and to ensure that the wearer has awareness of the environment around him or her at all times. It should be understood that the portion 142 of the display screen 108 having zoom-in functionality can be of any size or shape and can be located centrally, in an upper left quadrant, in an upper right quadrant, in a lower left quadrant, in a lower right quadrant, or in any other desired portion of the display screen 108 so long as there is at least some periphery 168 present to permit the wearer to have a view of the real-world, real time video feed 174. In some embodiments, the portion 142 of the display screen 108 with zoom-in functionality and occupy from about 5% to about 95%, such as from about 10% to about 90%, such as from about 25% to about 75% of the inner surface 166 of the lens 138.

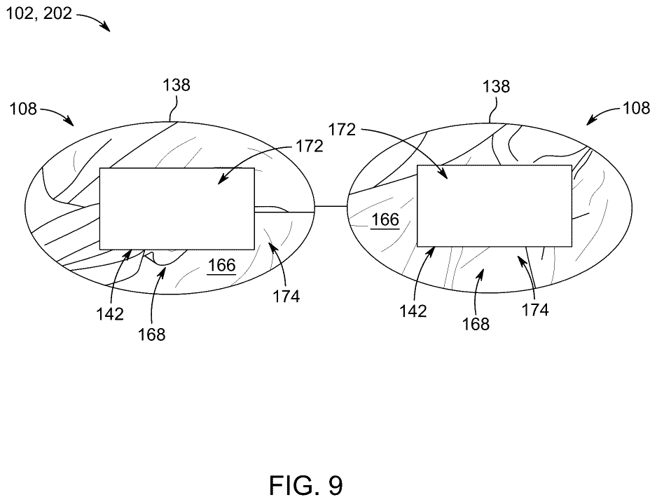

Next, FIG. 9. illustrates a display screen 108 on an inner surface 166 of two lenses 138 of a head-mounted display 102 or 202, where both lenses 138 show a live, real-world video feed 174 without zoom-in from the perspective of the wearer of the first head-mounted display 102 or a wearer of a second head-mounted display 202 about a periphery 168 of the display screen 108, while the portion 142 of the display screen 108 with zoom-in functionality on each lens 138 is shown in dashed lines and includes a zoomed-in image 172.

Turning now to FIG. 10, a computer system 300 that can be used in conjunction with or as a part of the XR head-mounted display systems 100 and 200 of the present disclosure is shown in accordance with one aspect. The computer system 300 can be an electronic computer framework comprising and/or employing any number and combination of computing devices and networks utilizing various communication technologies, as described herein. The computer system 300 can be easily scalable, extensible, and modular, with the ability to change to different services or reconfigure some features independently of others. The computer system 300 may be, for example, a server, desktop computer, laptop computer, tablet computer, or smartphone that is used to enable the functionality and transfer of data between the XR head-mounted display systems 100 and 200 of the present disclosure. In some examples, computer system 300 may be a cloud computing node. Computer system 300 may be described in the general context of computer-executable instructions, such as program modules, being executed by a computer system. Generally, program modules may include routines, programs, objects, components, logic, data structures, and so on that perform particular tasks or implement particular abstract data types. Computer system 300 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 10, the computer system 300 has one or more central processing units (CPU(s)) 301a, 301b, 301c, etc. (collectively or generically referred to as processor(s)). The processors can be a single-core processor, multi-core processor, computing cluster, or any number of other configurations. The processors can be any type of circuitry capable of executing instructions. The processors, also referred to as processing circuits, are coupled via a system bus 302 to a system memory 303 and various other components. The system memory 303 can include one or more memory devices, such as read-only memory (ROM) 304 and a random-access memory (RAM) 305. The ROM 304 is coupled to the system bus 302 and may include a basic input/output system (BIOS), which controls certain basic functions of the computer system 300. The RAM is read-write memory coupled to the system bus 302 for use by the processors. The system memory 303 provides temporary memory space for operations of said instructions during operation. The system memory 303 can include random access memory (RAM), read-only memory, flash memory, or any other suitable memory systems.

The computer system 300 comprises an input/output (I/O) adapter 306 and a communications adapter 307 coupled to the system bus 302. The I/O adapter 306 may be a small computer system interface (SCSI) adapter that communicates with a hard disk 308 and/or any other similar component. The I/O adapter 306 and the hard disk 308 are collectively referred to herein as a mass storage 310.

Software 312 for execution on the computer system 300 may be stored in the mass storage 310. The mass storage 310 is an example of a tangible storage medium readable by the processors 301a, 301b, and 301c, where the software 312 is stored as instructions for execution by the processors to cause the computer system 300 to operate, such as is described hereinbelow with respect to the various Figures. Examples of computer program product and the execution of such instruction is discussed herein in more detail. The communications adapter 307 interconnects the system bus 302 with a network 126, which may be an outside network, enabling the computer system 300 to communicate with other such systems. In one aspect, a portion of the system memory 303 and the mass storage 310 collectively store an operating system, which may be any appropriate operating system to coordinate the functions of the various components shown in FIG. 10.

Additional input/output devices are shown as connected to the system bus 302 via a display adapter 314 and an interface adapter 316. In one aspect, the adapters 306, 307, 314, and 316 may be connected to one or more I/O buses that are connected to the system bus 302 via an intermediate bus bridge (not shown). A display 319 (e.g., a screen or a display monitor) is connected to the system bus 302 by a display adapter 315, which may include a graphics controller to improve the performance of graphics-intensive applications and a video controller. A keyboard, a mouse, a touchscreen, one or more buttons, a speaker, etc., can be interconnected to the system bus 302 via the interface adapter 316, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 10, the computer system 300 can include processing capability in the form of the processors 301a, 30b, and 301c, and storage capability including the system memory 303 and the mass storage 310, input means such as the buttons, touchscreen, and output capability including the speaker 324 and the display 319.

In some aspects, the communications adapter 307 can transmit data using any suitable interface or protocol, such as the internet small computer system interface, among others. As described above with respect to FIG. 1, the network 126 may be a cellular network, a radio network, a wide area network (WAN), a local area network (LAN), or the Internet, among others. An external computing device may connect to the computer system 300 through the network 126. In some examples, an external computing device may be an external web server or a cloud computing node.

It is to be understood that the block diagram of FIG. 10 is not intended to indicate that the computer system 300 is to include all of the components shown in FIG. 10. Rather, the computer system 300 can include any appropriate fewer or additional components not illustrated in FIG. 10 (e.g., additional memory components, embedded controllers, modules, additional network interfaces, etc.). Further, the aspects described herein with respect to computer system 300 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

Aspects disclosed herein may be a system, a method, and/or a computer program product at any possible technical detail level of integration. The computer program product may include a computer-readable storage medium (or media) having computer-readable program instructions thereon for causing a processor to carry out various aspects.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present disclosure may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language, such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present disclosure.

Aspects are described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a computer system, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects are described herein with reference to the related drawings. Alternative aspects can be devised without departing from the scope of this disclosure. Various connections and positional relationships (e.g., over, below, adjacent, etc.) are set forth between elements in the following description and in the drawings. These connections and/or positional relationships, unless specified otherwise, can be direct or indirect, and the present disclosure is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects, and/or parts of the aspects, disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium, such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), graphics processing units (GPUs), microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be understood that various modifications may be made to the embodiments disclosed herein. Therefore, the above description should be not construed as limiting, but merely as exemplifications of various embodiments. Those skilled in the art will envision other modifications within the scope and spirit of the appended claims, and the foregoing description is by way of example only. Thus, it is not intended to limit the invention so further described in the appended claims. In addition, it should be understood that aspects of the various embodiments may be interchanged both in whole or in part.

What is claimed is:

1. An extended reality head-mounted display system comprising:
a head-mounted display comprising a lens and a display screen located on an inner surface of the lens for projecting a real-world video feed;
integrated zoom-in functionality; and
a processor coupled to memory, wherein the processor is configured to execute instructions that cause a portion of the display screen to project a zoomed-in image of the real-world video feed to a wearer of the head-mounted display.

2. The extended reality head-mounted display system of claim 1, further comprising an input configured to allow the wearer of the head-mounted display to activate the integrated zoom-in functionality.

3. The extended reality head-mounted display system of claim 2, wherein the one or more inputs comprises a microphone, a physical actuator, or a combination thereof.

4. The extended reality head-mounted display system of claim 1, wherein the head-mounted display further comprises an eye-tracking sensor.

5. The extended reality head-mounted display system of claim 1, further comprising a camera, wherein the camera provides the real-world video feed to the display screen in real time.

6. The extended reality head-mounted display system of claim 5, wherein the camera is a part of the head-mounted display.

7. The extended reality head-mounted display system of claim 5, wherein the camera is an external camera that is separate from the head-mounted display.

8. The extended reality head-mounted display system of claim 1, wherein a periphery of the display screen is configured to display the real-world video feed when the integrated zoom-in functionality is activated.

9. The extended reality head-mounted display system of claim 1, wherein the real-world video feed includes a surgical procedure.

10. The extended reality head-mounted display system of claim 1, wherein the zoomed-in image captures a surgical procedure being performed on a patient, one or more vital signs of a patient, a surgical environment, or a combination thereof.

11. An extended reality head-mounted display system comprising:
a first head-mounted display comprising a first lens and a first display screen located on an inner surface of the first lens for projecting a first real-world video feed;
a first integrated zoom-in functionality associated with the first head-mounted display;
a first processor coupled to a first memory, wherein the first processor is configured to execute instructions that cause a portion of the first display screen to project a first zoomed-in image of the first real-world video feed to a first wearer of the first head-mounted display;
a second head-mounted display comprising a second lens and a second display screen located on a second inner surface of the second lens for projecting the first real-world video feed;
a second integrated zoom-in functionality associated with the second head-mounted display; and
a second processor coupled to a second memory, wherein the second processor is configured to execute instructions that cause a portion of the second display screen to project a second zoomed-in image of the first real-world video feed to a second wearer of the second head-mounted display.

12. The extended reality head-mounted display system of claim 11, wherein the first zoomed-in image of the first real-world video feed on the first display screen is not visible to the second wearer of the second head-mounted display, and wherein the second zoomed-in image of the first real-world video feed on the second display screen is not visible to the first wearer of the first head-mounted display.

13. The extended reality head-mounted display system of claim 11, further comprising one or more inputs configured to independently allow the first wearer of the first head-mounted display to activate the first integrated zoom-in functionality on the first head-mounted display and the second wearer of the second head-mounted display to activate the second integrated zoom-in functionality on the second head-mounted display.

14. The extended reality head-mounted display system of claim 11, wherein the first zoomed-in image of the first real-world video feed is visible to the second wearer so that the second zoomed-in image is a duplicate of the first zoomed-in image of the first real-world video feed.

15. The extended reality head-mounted display system of claim 11, wherein the first head-mounted display, the second head-mounted display, or both further comprise an eye tracking sensor.

16. The extended reality head-mounted display system of claim 11, further comprising a first camera that is part of the first head-mounted display, wherein the first camera projects the first real-world video feed to the first display screen, the second display screen, or both in real time.

17. The extended reality head-mounted display system of claim 11, further comprising a second camera that is part of the second head-mounted display.

18. The extended reality head-mounted display system of claim 11, wherein a periphery of the first display screen is configured to display the first real-world video feed when the first integrated zoom-in functionality is activated, and wherein a periphery of the second display screen is configured to display the first real-world video feed when the second integrated zoom-in functionality is activated.

19. A method comprising:
receiving, via a first head-mounted display comprising a first processor, a real-world video feed;
projecting, via the first head-mounted display, the real-world video feed onto a first display screen located on a first inner surface of a first lens of the first head-mounted display; and
receiving, via a first wearer of the first head-mounted display, a first input that causes the first processor to execute instructions to zoom-in on a portion of the real-world video feed being projected on the first display screen.

20. The method of claim 19, further comprising:
receiving, via a second head-mounted display comprising a second processor, the real-world video feed;
projecting, via the second head-mounted display, the real-world video feed onto a second display screen located on a second inner surface of a second lens of the second head-mounted display; and
receiving, via a second wearer of the second head-mounted display, a second input that causes the second processor to execute instructions to zoom-in on a portion of the real-world video feed being projected on the second display screen.
